SEQUENCE LISTING (5 sequences)

SEQ ID NO: 1 - DNA, 21 bp, synthetic construct
  source 1..21: mol_type = other DNA
ggagtgtggattcgcactcct

SEQ ID NO: 2 - DNA, 20 bp, synthetic construct
  source 1..20: mol_type = other DNA
agattgagatcttctgcgac

SEQ ID NO: 3 - DNA, 27 bp, synthetic construct
  source 1..27: mol_type = other DNA
aggcaggtcccctagaagaagaactcc

SEQ ID NO: 4 - DNA, 23 bp, synthetic construct
  source 1..23: mol_type = other DNA
ttaggcagaggtgaaaaaagttg

SEQ ID NO: 5 - DNA, 23 bp, synthetic construct
  source 1..23: mol_type = other DNA
ttaggcagaggtgaaaaaagttg

(19) United States
(12) Patent Application Publication
(10) Pub. No.: US 2024/0294880 A1
(43) Pub. Date: Sep. 5, 2024

(54) STABLE CLONAL CELL LINE AS A HBV RNA STANDARD

(71) Applicants: Universite Claude Bernard Lyon 1, Villeurbanne (FR); Centre National de la Recherche Scientifique, Paris (FR); INSERM (Institut National de la Santé et de la Recherche Médicale), Paris (FR); Hospices Civils De Lyon, Lyon (FR); Centre Leon Berard, Lyon (FR)

(72) Inventors: Massimo Levrero, Lyon (FR); Fabien Zoulim, Dardilly (FR); Alexia Paturel, Villeurbanne (FR)

(73) Assignees: Universite Claude Bernard Lyon 1, Villeurbanne (FR); Centre National de la Recherche Scientifique, Paris (FR); INSERM (Institut National de la Santé et de la Recherche Médicale), Paris (FR); Hospices Civils De Lyon, Lyon (FR); Centre Leon Berard, Lyon (FR)

(21) Appl. No.: 18/592,164

(22) Filed: Feb. 29, 2024

(30) Foreign Application Priority Data
Mar. 3, 2023 (EP) 23305284.4

(51) Int. Cl. C12N 5/09; C12P 19/34; C12Q 1/70; C12R 1/91

(52) U.S. Cl. CPC C12N 5/0693; C12P 19/34; C12Q 1/706; C12N 2510/02; C12R 2001/91

(57) ABSTRACT

The invention provides a clonal cell line derived from a liver cancer cell line, preferably derived from a Huh7 hepatocarcinoma cell line, engineered to produce and secrete viral particles containing pregenomic RNA of the hepatitis B virus (HBV), predominantly over DNA of HBV, to calibrate quantitative HBV RNA assays.

### CROSS-REFERENCE TO RELATED APPLICATIONS

This application claims priority to European Patent Application No. 23305284.4, filed on Mar. 3, 2023, the disclosure of which is hereby incorporated by reference in its entirety.

### REFERENCE TO A SEQUENCE LISTING

The sequence listing of the present application is submitted electronically as a computer readable sequence listing in XML format with a file name of “PR2721.xml,” a creation date of Feb. 27, 2024, and a size of 5,015 bytes. The sequence listing submitted is part of the specification and is hereby incorporated by reference in its entirety.

### TECHNICAL FIELD

The invention relates to a stable clonal cell line to produce a hepatitis B virus (HBV) RNA standard as a calibrator for HBV RNA quantification assays.

### BACKGROUND OF THE INVENTION

HBV remains a major public health problem worldwide, despite the availability of an effective vaccine and antiviral therapies. More than 240 million people are chronically infected, with 0.5-1 million dying annually because of HBV-related advanced liver disease. Long term treatment with anti-HBV nucleos(t)side analogues (NUCs) suppresses viral replication, liver inflammation and disease progression. However, only a small proportion of patients achieve a functional HBV cure, defined by a sustained off-treatment loss of HBsAg that is associated with an improved long-term clinical outcome (remission of liver inflammation, decrease in the risks of cirrhosis and HCC) and can stop treatment with very low rates of disease reactivation. Innate and adaptive antiviral immune responses control this residual reservoir of viral minichromosome reservoir. A complete HBV cure with eradication of all covalently closed circular DNA (cccDNA) in the liver and no residual risk of viral reactivation, is rarely, if at all, reached with NUCs. The development of new antiviral therapies targeting the cccDNA reservoir is a major objective towards HBV cure. Several new molecular entities (NMEs) with direct antiviral or immuno-modulatory activities, are at the preclinical or clinical evaluation stage with the aim of inducing a functional cure with a time-limited treatment.

Direct assessment of cccDNA levels and activity requires liver biopsy, an invasive procedure, and cccDNA quantification remains technically challenging. To assist the clinical development of these NMEs, there is an urgent need for circulating biomarkers that adequately reflect the size and activity of intrahepatic cccDNA. HBV DNA and HBsAg, the currently used biomarkers to define HBV endpoints have several limitations in this setting: i) HBV DNA in scrum is the best indicator of the antiviral activity of a new treatment in naive patients but it is already suppressed in NUC treated patients; ii) HBsAg loss cannot be considered a biomarker of the activity of new therapeutic molecules since it is at the same time the desired endpoint and part of the definition of cure; iii) the predictive value of different thresholds of HBsAg decrease remains to be validated and the kinetic of HBsAg decrease is often too slow to allow early outcome prediction in clinical trials. In addition, HBsAg may derive from the integration of the viral genome into the host genome and not fully reflect the transcriptional activity of cccDNA. New biomarkers would also be useful: a) to better characterize patients in the different phases of the HBV infection and disease; b) to predict response to interferon (IFN) at an early stage allowing to extend the treatment only in patients with high probability to reach a functional cure; c) to identify patients that could discontinue NUCs without risk of relapse; d) to assess target engagement and antiviral activity in patients receiving NMEs; c) to predict HBsAg response, i.e. functional cure, in patients receiving NMEs.

Several studies have identified circulating HBV RNAs as a biomarker reflecting the transcriptional activity of cccDNA and have shown that circulating HBV RNA may help monitor CHB infection. The quantification of HBV pregenomic RNA expressed from cccDNA and not from integrated viral DNA, is a robust marker of cccDNA transcriptional activity. Although the potential of circulating HBV RNAs as a biomarker is well established, a major limitation is represented by the lack of standardized protocols (e.g., samples extraction, retro-transcription, choice of primers for amplification, genotype inclusivity). Investigational “research only” kits to quantify circulating HBV RNAs have been developed by industry (Butler et al., 2018) (Scholtès et al. 2022). A second important limitation is the lack of a recognized standard to calibrate circulating HBV RNA quantification.

A World Health Organization (WHO) International Standard (IS) for HBV DNA has been established to use as a calibrator for HBV nucleic acid amplification technique (NAT)-based assays. Due to their limited amount, four standards, derived from the same plasma donor (genotype A2, HBsAg subtype adw2, quantified at 2.7×10⁹ HBV DNA molecules/mL (Heermann et al., 1999), GenBank accession number KY003230 (Jenkins et al., 2017)) have been validated and distributed over time (Baylis et al., 2008; Fryer et al., 2017; Organization and Standardization, 2016; Saldanha et al., 2001).

The WHO HBV DNA standard has been used as a calibrator for the quantification of serum HBV RNA (Butler et al., 2018) but its appropriateness has been challenged. In particular, it is questionable whether an HBV RNA unit is equivalent to an international HBV DNA unit defined by the plasma derived WHO HBV DNA standard that is contaminated by HBV RNA (Liu et al., 2020).

### SUMMARY OF THE INVENTION

The inventors now provide a stable clonal cell line producing an RNA-based standard that is particularly useful for circulating HBV RNA assays calibration.

A subject of the invention is a cell line derived from a liver cancer cell line, preferably derived from a Huh7 hepatocarcinoma cell line, engineered to produce and secrete viral particles containing pregenomic RNA of the hepatitis B virus (HBV), predominantly over HBV DNA.

In a preferred embodiment, the cell line is the clonal cell line derived from a Huh7 hepatocarcinoma cell line and deposited under Budapest Treaty at COLLECTION NATIONALE DE CULTURES DE MICROORGANISMES (CNCM), Institut Pasteur, 25-28 rue du Docteur Roux, 75015, Paris, France, on Aug. 1, 2022 under deposit number CNCM I-5878. It is also named “Huh7-3D29” or “3D29” in the present disclosure.

The invention further provides a vector, preferably a plasmid vector, that comprises at least a 1.1×HBV DNA genome carrying mutations that alter viral DNA synthesis but preserve pregenomic RNA synthesis and viral protein expression, preferably wherein the mutations comprise, or consist, of: i) D540A and D541A amino acid changes in the catalytic region of Pol ORF, and ii) Y63F amino acid change in the TP-domain of the Pol ORF.

In a preferred embodiment, the cell line of the invention comprises a HBV genome that carries mutations that comprise, or consist, of i) D540A and D541A mutations in the catalytic region of Pol ORF, and ii) Y63F mutation in the TP-domain of the Pol ORF.

A further subject of the invention is a method for producing HBV RNA particles, which method comprises: i) culturing the cells as defined herein in a culture medium and under conditions that allow the cells to secrete the HBV RNA particles, and ii) collecting the supernatant(s), that comprise HBV RNA particles.

This standard operating procedure (SOP) allows to generate standard supernatants for calibration of HBV RNA assays.

The supernatants or collection of supernatants obtained or obtainable by this method or SOP are also encompassed in the present invention.

Kits for calibrating HBV RNA assays are provided, comprising at least one container with HBV RNA containing particles in a supernatant or collection of supernatants, preferably wherein the HBV RNA containing particles are in a concentration of at least 10⁷ copies/ml.

The supernatant or collection of supernatants are useful as a standard for calibration of circulating HBV RNA quantification assays, e.g., for clinical or investigational use.

Said supernatant or collection of supernatants or said kit, is useful in an in vitro method for diagnosing or monitoring an infection by HBV, assessing or monitoring a HBV treatment, or for predicting patient outcome. In particular, it is further provided an in vitro method for diagnosing or monitoring an infection by HBV, assessing or monitoring a HBV treatment, or for predicting patient outcome, which method comprises determining or quantifying HBV RNA by means of said supernatant or collection of supernatants or said kit.

### LEGENDS TO THE FIGURES

FIG. 1. 3D29 HBV RNA standard validation using the HBV RNA Roche manual workflow (MWF) assay.
(A) Quantification of ten folds dilution (from 10⁵ to 10¹ copies/mL) of HBV armored RNA (arRNA, Roche Diagnostics, Pleasanton, CA) (left panel (i)) and Huh7-3D29 supernatants (right panel (iii)), processed as described in Examples below, using the HBV RNA Roche MWF kit. Ct values and corresponding concentration in copies/mL are given in the middle panel (ii). No significant difference was found between Ct of 10-fold diluted armored RNA and Huh7-3D29 RNA using a paired t test (P-value=0.54). Validation of samples as parametric by Shapiro-Wilk test. (B) 3D29 HBV RNA quantification with and without DNAse treatment on 3 different passages of 3D29. No significant difference was found between DNAse treatment and no DNAse treatment using a Wilcoxon test (P-value=0.25). Validation of samples as nonparametric by Shapiro-Wilk test.

FIG. 2. HBV DNA and HBV RNA compartmentalization in Huh7-3D29 cell supernatant.

Analysis of (A) Huh7 cell clone transfected with wild type (WT) HBV genome named Huh7-WT18 and (B) Huh7-3D29 cells supernatants by iodixanol/sucrose density gradient ultracentrifugation. (i) HBsAg quantification by ELISA, HBV DNA, and total HBV RNA quantification by ddPCR in gradient fractions. (ii) Western Blot detection of viral HBc protein and CD9 exosome marker.

FIG. 3—Characterization of HBV RNAs from Huh7-3D29 cell culture supernatants. Oxford Nanopore long reads sequencing of 5′ RACE products from 3D29 cells supernatants (A) and 3D29 intracellular RNAs (B). The majority of HBV RNAs detected are the full length transcript and the pgRNA derived spliced RNAs; only a minority of PreS/S RNAs, PreS/S derived spliced and HBx RNAs were detected.

### DETAILED DESCRIPTION OF THE INVENTION

More generally, the cell line according to the invention is a clonal cell line, that is stable.

It is in isolated form.

The cell line of the invention derives from a liver cancer cell line, which may be a hepatocarcinoma or a hepatoblastoma cell line.

It preferably derives from a Huh7 hepatocarcinoma cell line, however other cell lines may be used, e.g., HepG2, Huh6.

In a preferred embodiment, the cell line of the invention was engineered to integrate a HBV genome that carries mutations at least the following mutations: i) D540A and D541A mutations in the catalytic region of Pol ORF, and ii) Y63F mutation in the TP-domain of the Pol ORF.

As the most preferred embodiment, the stable clonal cell line 3D29 that derives from a Huh7 cell line, carries a stable HBV integrant with a double mutation in the catalytic site of the polymerase (YMAA) and Y63F mutation in the TP-domain of the Pol ORF and shows the following properties:
i) it secretes high amounts of HBV RNAs with low quantities of HBV DNA;
ii) it shows the same compartmentalization of secreted HBV RNA observed in vivo (namely naked capsids, virions-like particles, exosomes);
iii) it shows a stable phenotype (a high RNA versus DNA secretion ratio) over serial passages in culture;
iv) it allows for high productivity (one 175 cm³ flask of cells produces enough supernatant to perform more than 1,000 standard curves).

In a preferred embodiment, the cell lines of the invention may be generated by transfection with a vector, preferably a plasmid vector, that comprises a 1,1×HBV DNA genome carrying mutations that alter viral DNA synthesis but preserve pregenomic RNA synthesis and viral protein expression, resulting in the production and secretion of viral particles containing pregenomic RNA of HBV without substantial secretion of viral DNA containing particles.

The 1,1×HBV DNA genome may be of any viral genotype. In a particular embodiment, the HBV DNA genome is of genotype D.

The HBV genome is 3.2 kb and circular. The full-length pgRNA is 3.5 kb and linear. The polymerase makes more than one circle when it is transcribed from the natural template (e.g., the cccDNA) that is circular. To obtain the pgRNA from a linear integrated HBV DNA, one should integrate (in the transfected and clonally selected cells) an HBV DNA that is longer (at least 1.1 or 1.2×) than the full 3.2 kb HBV genome. In a particular embodiment, two units of the genome may be used.

Pregenomic RNA (pgRNA) has a size of about 3.5 kb. In HBV natural infection, the pgRNA is transcribed from cccDNA but not from integrated viral DNA and it is translated into HBV proteins (i.e. core and pol) and/or reverse-transcribed into HBV DNA.

The cell line allows for production and secretion of viral particles containing pregenomic RNA of HBV without substantial secretion of viral DNA containing particles. This means that the cell line allows for predominant secretion of viral particles containing pregenomic RNA of HBV over viral DNA containing particles.

In a preferred embodiment, the ratio of HBV secreted RNA (copy number)/HBV DNA (copy number) is at least 25/1.

Vectors that comprise the mutated HBV DNA genome may be e.g. a non-viral vector, preferably a DNA plasmid. Typically, the vector may further comprise regulatory sequences. As used herein, the term “regulatory sequence” refers to any sequence that allows, contributes or modulates the functional regulation of the nucleic acid molecule, including replication, transcription, splicing, translation, stability and/or transport of the nucleic acid into the host cell.

Transfection of a host cell or cell line can be achieved by any method known by the skilled person.

The cell line may then be cultured in a culture medium and under conditions that allow the cells to secrete the HBV RNA particles. The supernatant(s) that comprise HBV RNA containing particles may then be collected.

In a particular embodiment, the cells are cultured during at least 6 days, preferably at least 9 days.

Suitable culture media are known to the skilled person. As an example, DMEM may be employed.

The cell lines may be stored under appropriate conditions for cell freezing known to the skilled person.

The supernatant or collection of supernatants may also be stored for further use, e.g. in a liquid form, freeze-dried or lyophilized form.

The invention provides a container, e.g. a flask or tube, containing such supernatant or collection of supernatants.

The present disclosure also provides for methods of detecting the presence or absence of HBV RNA, in a biological sample from an individual, using the HBV RNA particles produced by the SOP method described herein. Such methods generally include performing at least one cycling step, which includes an amplifying step and a dye-binding step.

These methods can be employed to detect the presence or absence of HBV RNA (in particular, pgRNA) in plasma, e.g., for use in blood screening and diagnostic testing. Additionally, the same test may be used by someone experienced in the art to assess other sample types to detect and/or quantitate HBV RNA (in particular, pgRNA). Such sample types can include any such sample, where HBV RNA, may be found, including whole blood, serum, biopsy samples, fine needle aspirates, urine. . .

In an embodiment, a kit for calibrating HBV RNA assays is provided which comprises at least one container with HBV RNA particles in a supernatant or collection of supernatants as defined herein.

In a preferred embodiment, the HBV RNA particles are in a concentration of at least 10⁷ copies/ml.

It is also provided a kit for detecting and/or quantitating HBV RNA (in particular, HBV pgRNA) which, in addition to the calibration container containing HBV RNA particles in a supernatant or collection of supernatants, may also include one or more primers or sets of primers specific for amplification of the gene target; and one or more detectable oligonucleotide probes specific for detection of the amplification products.

Quantification of HBV RNA may be applied to several uses, in particular in diagnosing or monitoring an infection by HBV, for diagnosing or monitoring an infection by HBV, assessing or monitoring a HBV treatment, or for predicting patient outcome. Other applications encompass research purposes with the ex-vivo study of HBV infection in biological samples from HBV infected animals or humans, as well as in vitro investigations of HBV infection in cell culture.

The invention provides a standard for calibration of circulating HBV RNA quantification assays, in any clinical and or investigational use.

The Examples and Figures illustrate the invention without limiting its scope.

### EXAMPLES

### Example 1: Generation of a Molecular Standard for Circulating HBV RNA Detection and Quantification Assays in Patients with Chronic Hepatitis B (CHB)

### Materials and Methods

### Cell Lines

The human Huh7 hepatocarcinoma cell line was cultured at 37° C. in a humidified atmosphere containing 5% CO2 in Dulbecco's modified Eagle medium supplemented with 10% Hyclone fetal clone II serum 1% GlutaMax (Gibco), 1% Penicillin/Streptomycin (Gibco), 1% sodium pyruvate (Gibco), 1% non-essential amino acids (NEAA, Gibco).

### Plasmids

The HBV RNA producing cell lines were generated by transfecting a pTriEX plasmid derivative containing 1.1 length wild type and mutated HBV DNA genomes in Huh7 cells. The following mutations were selected: D540A and D541A mutations in the catalytic domain of Pol ORF (i.e. YMDD motif mutated into a YMAA amino acid sequence), and Y63F mutation in the TP-domain of the Pol ORF. HBV mutated genomes of genotype D have been generated and inserted into the pTriEX-Bsd vector: a plasmid containing the WT 1.1 HBV DNA, 2 plasmids containing the 1.1 HBV DNA carrying the YMAA mutation or the Y63F mutation and a plasmid containing the 1.1 HBV DNA carrying the following combination of mutations: YMAA+Y63F. All plasmids include the blasticidin gene to confer resistance. Wild type and mutated 1.1 HBV DNA genomes were synthetized and the plasmids produced and controlled by sequencing by GenScript®.

### Cell Transfection and Culture

Huh7 cells were seeded at 60-80% confluency and were allowed to adhere overnight. Cells were then transfected with the indicated amounts of total plasmid DNA with the TransIT®-2020 kit (Mirus) in serum-free Opti-MEM medium (Life Technologies), following the manufacturer's instructions. After 12h, the transfection medium was removed and was replaced with fresh medium. Transfected cells were grown and amplified in selective growth media (Huh7 medium described above) supplemented with 4 μg/mL of Blasticidin).

Clonal Cell Line Isolation and Expansion

Colony formation was obtained after serial dilution of Huh7 polyclonal cell population from 1×10⁴ cells to 100 cells per 150 cm² petri dish. Cell plates were incubated at 37° C. with 5% CO₂ for 20 days to allow colony formation and growth. Using single channel pipettor, single colonies were transferred to 1 mL PBS. Cells were pelleted by centrifugation for 5 min at 900 rpm and resuspended in 50 μL of 0.25% trypsin-EDTA (Gibco) and kept for 5 min at 37° C. Cells were then plated in 24 wells plates with 1 mL fresh Huh7 media. Clonal cell lines were amplified under blasticidin selection. Clones to be further carried on were chosen based on cell growth, HBV RNA/HBV DNA ratio.

### HBV RNA Supernatants Production

To test the stability over time of the HBV RNA secretory phenotype and to perform higher scale supernatants collection for the production of the HBV RNA standard, cells were seeded (25×10⁶ cells/175 cm² flask) and left to adhere overnight before change of media containing 2.5% DMSO. To produce large quantities of HBV RNAs, cells were kept under standard culture conditions for 9 days. Every 3 days, supernatant was collected, and fresh media was added. At the end of the 9 days of cultures, all collected supernatant were pooled and centrifuged for 5 min at 1,500 rpm to remove any cellular debris.

### Ultracentrifugation, DNA and RNA Isolation

Pooled cell culture supernatants were ultracentrifuged for 5 hours at 25,000 rpm over a 4 mL 20% w/v sucrose cushion. After ultracentrifugation, the supernatant was removed, and the virus-containing pellet was resuspended in 600 μL of PBS 1X. 28.5 μL aliquots (28.5 μL out of 600 μL) of each resuspended pellet was completed to 200 μL with PBS 1X. DNA and RNA from supernatants were extracted using High Pure Viral Nucleic Acid (Roche, Diagnostics) according to the manufacturers' instructions. The same final volume of extracted nucleic acid elution (40 μL) was used for DNA or RNA analysis. RNA samples were treated with RQ1 RNase-Free Dnase (Promega, Cat #M6101) for 30 min at 37° C. and stored until use.

Reverse Transcription (RT) and Droplet Digital PCR (ddPCR)

For HBV RNA analysis, 4 μL of DNAse-treated RNA was reverse transcribed and amplified using the SuperScript™ IV VILO™ Master Mix (Invitrogen, Cat #11766500). A 22 μL reaction mixture was prepared comprising 11 μL of 2X ddPCR Supermix™ for probes (no dUTP) (Bio-Rad), 1.1 μL of primers and probe mix, and 5 μL of cDNA or DNA. Nucleic acid inputs were adjusted to have acceptable rates of negative events: samples were diluted 1:5. Probes and primers include: HBV (Pa03453406_s1, Thermofischer) for total HBV RNA quantification and pgRNA (Forward primer ggagtgtggattegcactcct SEQ ID NO:1, reverse primer agattgagatcttctgcgac SEQ ID NO: 2 and probe aggcaggtcccctagaagaagaactcc, SEQ ID NO:3) for pgRNA quantification. Droplet formation was carried out using a QX100 droplet generator. Subsequent amplifications were performed in the C1000 Touch™deep-well thermal cycler (Bio-Rad) with a ramp rate of 2° C./s and the lid heated to 105° C., according to the Bio-Rad recommendations. First, the enzyme was activated at 95° C. for 10 min followed by 40 cycles of denaturation at 94° C. for 30 s and 60° C. for one minute. The enzyme was deactivated at 98° C. for 10 min and the reaction was kept at 4° C. HBV RNA quantification was calibrated using a synthetic armored RNA (arRNA) containing 435 bp derived from the 3′ end of HBV pgRNA, packaged in MS2-phage (kindly provided by Roche Diagnostics, Pleasanton, CA). HBV RNA quantification with the MWF was performed according to the manufacturer's protocol (Scholtes C, 2022).

### 5′RACE Analysis

5′RACE was performed as previously described in Stadelmayer et al., 2020. Briefly, RNAs were isolated using a guanidinium thiocyanate-phenol-chloroform extraction protocol (TRI reagent (Sigma). 5′RACE was essentially performed as described in the GeneRacer Kit manual (ThermoFisher Scientific) except Tobacco Acid Pyrophosphatase was substituted by RNA 5′ Pyrophosphohydrolase (New England Biolabs) and SuperScript reverse transcriptase III by SuperScript reverse transcriptase IV (ThermoFisher Scientific). The reverse transcription reaction was performed using 3′ HBV specific Gsp1 primer 5′-TTAGGCAGAGGTGAAAAAAGTTG-3′ (SEQ ID NO:4). For the 5′RACE PCR reaction Prime Star super mix DNA Polymerase (TAKARA bio), GeneRacer 5′ primer and HBV specific nested primer Gsp1 5′-TTAGGCAGAGGTGAAAAAAGTTG-3′ (SEQ ID NO:5) were used. 5′RACE PCR was run in a C100 Touch thermocycler (Biorad) using the following touch down PCR program: Initial denaturation step 98° C. 3 min>5x (98° C. 10 s; 72° C. 3 min) >5x (98° C. 10 s; 70° C. 3 min)>25x (98° C. 10 s; 64,4 20 s; 72°° C. 3 min)>72° C. 10 min.

### Iodixanol/Sucrose Density Gradient Centrifugation

300 ml of supernatants were collected and centrifuged at 1500×g for 15 min at room temperature. Cellular debris were removed by filtration through a 0.22 μm filter (Merck Millipore, KGaA, Darmstadt, Germany). Viral particles were concentrated using Amicon® Pro Purification System with 100 kDa filter. Concentrated supernatants were then ultracentrifuged at 110,000×g for 2 h at 4°° C. The pellets were washed with 8 mL of PBS and a second ultracentrifugation was performed at 110,000×g for 2h at 4° C. Pellets were resuspended in 2.5 mL of 10% iodixanol solution. 10%, 20%, 30% and 40% iodixanol solutions were prepared by mixing Optiprep™ (Axis Shield) with buffer containing 0.25 M sucrose, 10 mM Tris at pH 8.0, and 1 mM EDTA, with final pH 7.4. Resuspended EVs pellets were layered on the top of the gradient and then subjected to ultracentrifugation in a SW41-Ti Rotor tube (Beckman) for 6 hours at 4°° C. at 110,000×g. Twelve fractions of 1 ml were recovered and analyzed separately.

### Elisa Tests

ELISA tests for HBeAg and HBsAg detection in cell supernatants were performed according to the manufacturer's protocol using the CLIA kits from Autobio Diagnostic.

### Western Blot

Fractions from density gradient were mixed with Laemmli buffer and heated at 95° C. for 5 min. Proteins were migrated in 4-20% mini-PROTEAN® TGX stain-Free™ Precast Gel (Bio-Rab Laboratories) and transferred onto a nitrocellulose membrane (Bio-Rab Laboratories). Membranes were blocked 1 hour with 5% milk or BSA (Sigma) in TBS (1×Tris Buffer Saline (Sigma)) and stained with primary antibodies in blocking buffer overnight at 4° C. and stained with HRP-conjugated secondary antibodies ( 1/50000) for 1 hour at room temperature. Detection was performed using Clarity or Clarity Max Western ECL and the ChemiDoc XRS system (Biorad).

### Libraries Preparation and MinION Sequencing

Products of 5′RACE PCR amplification performed with Prime Star super mix DNA Polymerase (TAKARA bio) were prepared for ONT MinION Sequencing using the SQK-PBK004 kit, according to the manufacturer's instructions. Sequencing was controlled and data were generated using ONT MinKNOW software (v3.4.12). Runs were terminated after 48 h and FAST5 files were generated.

### Data Processing and Bioinformatic Analysis

Base calling was performed with Guppy (v. 5.0.14, ONT) and simultaneously filtered for base called reads with PHRED quality >7. Further quality control of reads before and after mapping were performed with pycoQC (v. 2.5.2). Median PHRED score is 11.455 for passed reads and 99.6% of reads are conserved with acceptable quality. Reads containing amplification adapters sequences were extracted with seqkit (v. 2.1.0). The grep function permitted to extract reads containing Forward adapter or Reverse adapter, allowing 4 mismatches due to error rate of Oxford MinION Nanopore sequencing. Then, the rmdup function was used to remove duplicated reads. Reads that passed all filters were mapped to each known HBV transcript species (preC, pgRNA, preS1, preS2, HBs, L-HBx, canonical HBx, s-HBx), to splice-variants (SP01-20) derived from HBV genome (strain ayw, NC_003977.2), and to the genomic sequence corresponding to the full-length preCore RNA sequence (from TSS to the canonical HBV poly-A) with Minimap2 (v. 2.21-r1071) using the spliced long reads option (-ax splice). Sam files were filtered, binarized and sorted with Samtools (v. 1.7). HBV transcript species and splice variants were quantified using Salmon (v. 1.6.0) based on transcriptomic alignment. Start positions were extracted from genomic alignment using Samtools and graphical representations were produced using R scripts (v. 4.1.2).

### Statistics

Statistical tests were performed using GraphPad Prism v7.05 sofware. Pairwise comparisons were analyzed using the Wilcoxon signed-rank test for non-parametric data and paired t test for parametric data. Shapiro-Wilk normality test was performed to assess the normality of the parameter's distribution (Graphpad prism). Differences were considered significant at confidence levels greater than 95% (P≤0.05).

### Results

### Strategies to Generate an HBV RNA Standard

A wild type (WT) and 3 mutant 1.1 HBV genotype D genomes have been synthesized and cloned into a pTriEX vector plasmid. After transfection and blasticidin selection, 6 Huh7 cells polyclonal cell lines (WT, YMAA; Y63F; Y63F+YMAA). HBV DNA and HBV RNA analysis in cell extracts and cell supernatants showed that the polyclonal cell line carrying the Y63F+YMAA displayed the desired secretory phenotype with an inversion of the HBV DNA/RNA ratio in cell supernatant and was selected for clonal cell lines isolation.

The Huh7 polyclonal wild type (WT), and mutated Y63F+YMAA cell lines were plated at low density for colony formation. After 20 days of blasticidin selection 24 clones for each polyclonal cell line were isolated, expanded and phenotyped for HBV DNA/RNA secretion. The wild type clone Huh7-WT18, and the cell clones harboring mutated Y63F+YMAA HBV genome (Huh7-5D21, Huh7-3D29 and Huh7-3D35) were selected based on cell growth, HBV RNA/HBV DNA ratio and carried on for further characterization and expansion.

### Secretory Phenotype of Selected Clonal Cells Lines.

Huh7-3D29 clonal cell line showed, 9 days post plating, high HBV RNA levels in the cell supernatant as compared to HBV DNA levels. Huh7-WT18 cells secreted 9.3-fold more HBV DNA than HBV RNA (1.06×10⁷ copies of DNA/mL vs 0.7×10⁶ copies of RNA/mL). Conversely, the selected mutant cell line displayed an inversion of the secreted DNA/RNA ratio. The difference between secreted RNA and DNA levels was 5.6×10⁷ copies of RNA/mL in Huh7-3D29 cells. The mutant clonal cell line Huh-3D29 secreted 65 times more HBV RNA than the Huh7-WT18 wild type clone.

After first series of freezing/thawing (3 passages of amplification) all Huh7-3D29 mutant cells maintained the RNA secretory phenotype. A decrease in HBV nucleic acids secretion was observed over the first passages with a 34% reduction of total HBV RNA secretion for Huh7-3D29 cells but without a significant change in the HBV RNA/DNA ratio, that remained constant over time. Huh7-3D29 was confirmed to be the best candidate clonal cell line for HBV RNAs secretion.

The inverted HBV DNA/RNA ratio was confirmed also after a second cycle of freezing/thawing (3-4 passages of amplification). A decrease of 39% of total secreted HBV RNAs was observed in Huh7-3D29 cell line (5.57×10⁷ vs 3.39×10⁷ copies/mL) again the strong HBV RNA secretory phenotype was conserved.

### 3D29 HBV RNA Standard Validation Using the HBV RNA Roche MWF

Ten folds dilution (from 10⁵ to 10¹ copies/mL) of HBV armored RNA (arRNA, Roche Diagnostics, Pleasanton, CA) and Huh7-3D29 supernatants, processed as described above, have been quantified using the HBV RNA Roche MWF kit (FIG. 1). The amplification curves obtained after RT-qPCR from the MWF showed very similar profiles. Furthermore, no significant difference was found between CTs of the 10-fold diluted arRNA and Huh7-3D29 cell supernatants. HBV RNA quantification with and without DNase treatment on supernatants from Huh7-3D29 cells collected and processed at three different passages showed no significant difference. The Roche MWF used here is a manual version of the cobas® 6800/8800 automated investigational assay for HBV RNA quantification that, differently from the MWF, does not include a DNase treatment (Scholtes et al 2022). The lack of differences in RNA quantification with the MWF between +/−DNase treatments, validates the use of Huh7-3D29 supernatants as an RNA standard for the HBV RNA cobas® 6800/8800 automated assay.

### Characterization of the HBV RNAs Secreted by the Huh7-3D29 Clonal Cell Line

Huh7-WT18, Huh7-3D29, cells supernatants were further analyzed by iodixanol/sucrose density gradient ultracentrifugation followed by HBsAg quantification by ELISA, HBV DNA, and total HBV RNA quantification by ddPCR, HBc and CD9 detection by Western blot.

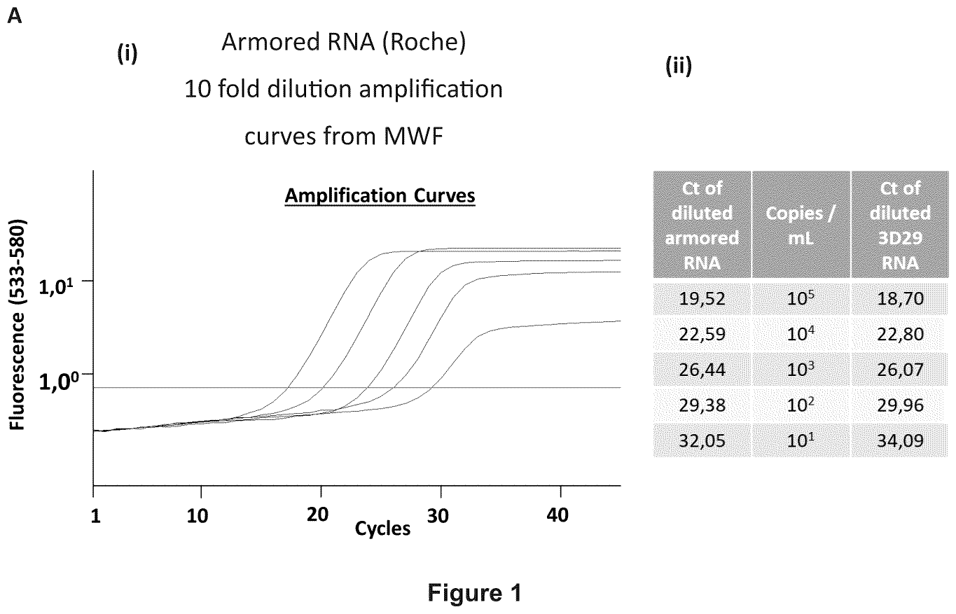

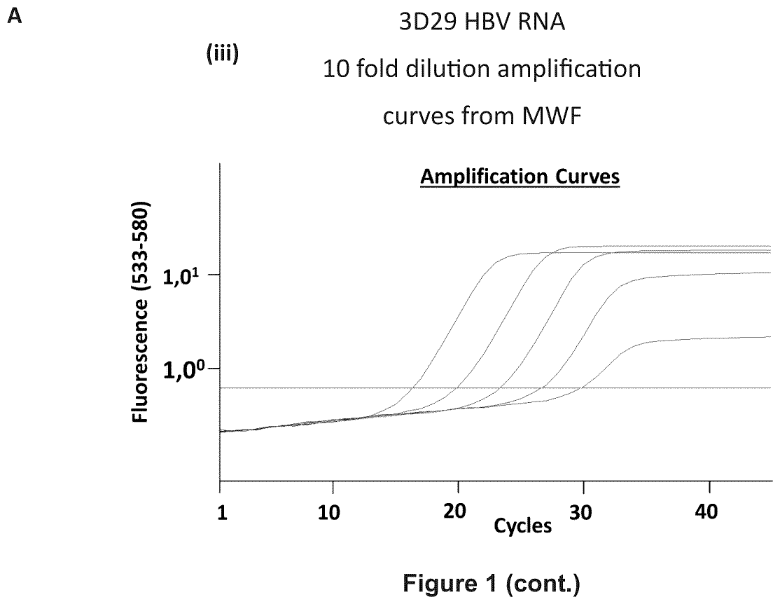

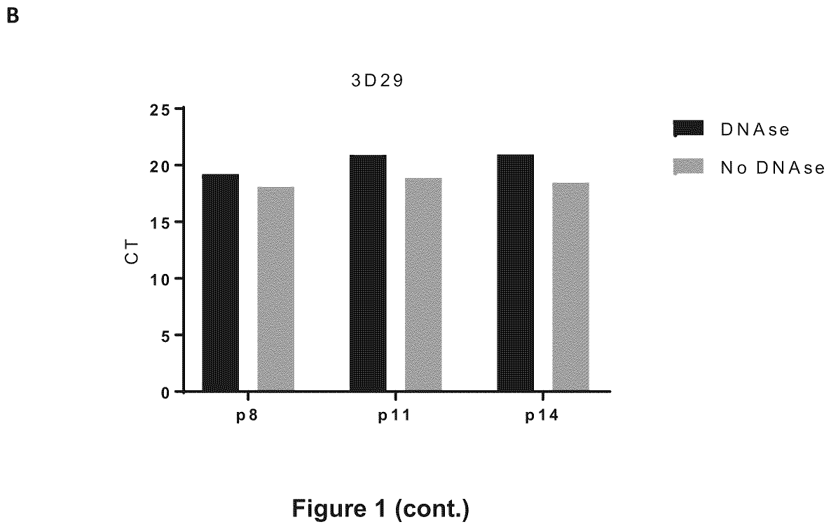

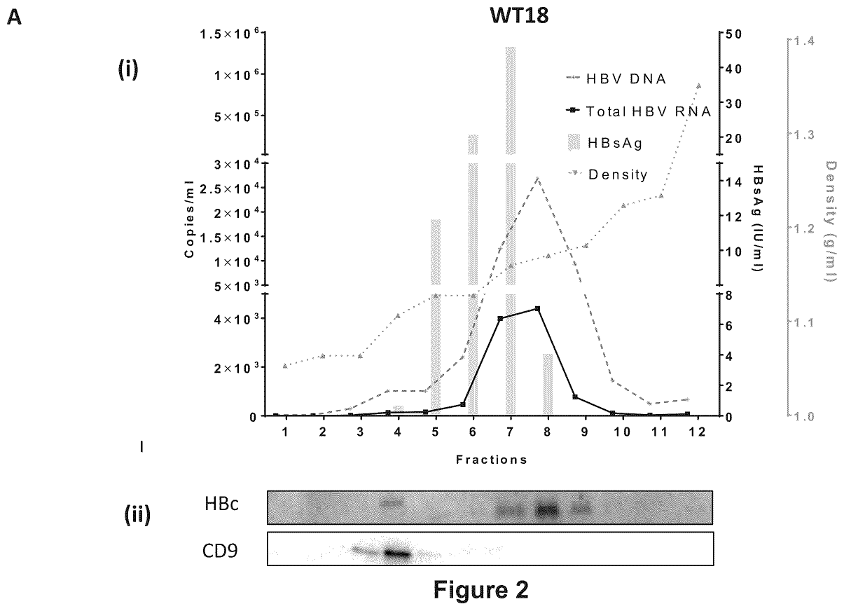

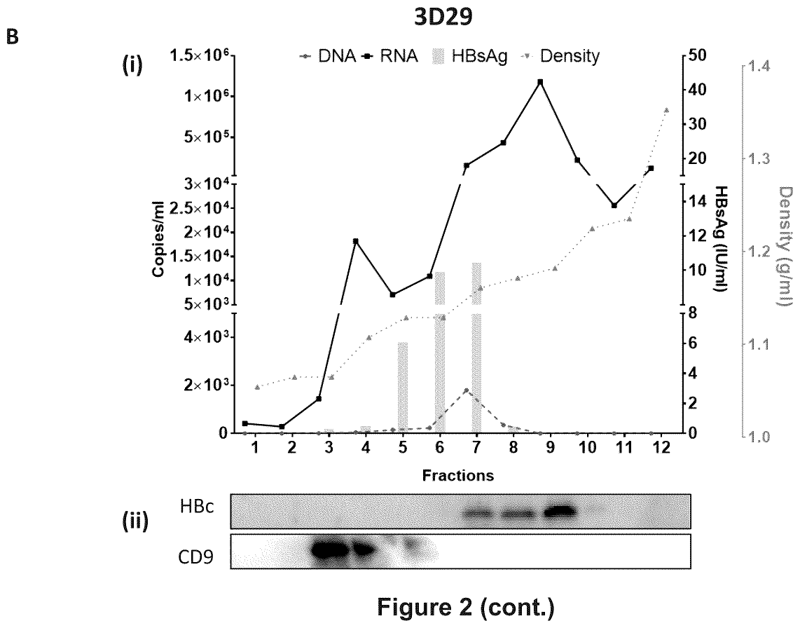

In Huh7-WT18 cell supernatants, HBsAg was detected in fractions 4-8 of the density gradient, with one major peak in fraction 7, and HBc (core protein) was detected from fractions 7 to 9 HBV DNA was found in almost all the fractions except in fractions 1 and 2, with a peak in fraction 8. HBV RNA is found in fractions 7-8 (FIG. 2). Fractions 3 and 4 contain exosomes, identified by the detection of the exosomal membrane protein CD9 (Klymiuk et al., 2019). In Huh7-WT18 cell supernatants exosomes contained only HBV DNA. The detection of both HBsAg and HBc identifies viral particles, while the detection of HBc only identifies naked capsids. Fractions 7 and 8, positive for both HBsAg and HBc, were positive for both HBV DNA (virions) and HBV RNA (RNA particles). Fraction 9, positive for HBc only, contained only low levels of HBV RNA compared to HBV DNA, indicating that naked capsids mostly carry HBV DNA in WT cells.

In the HBV mutant clonal cell line Huh7-3D29, the nucleic acids peak was shifted to fraction 7. The cell line presented a major HBV RNA peak in fraction 9. The observed shift of viral nucleic acids peak is relevant as the refractive index of the corresponding sucrose fraction for each isolation was almost identical, which excludes the possibility that the shift was caused by difference in sample preparation. Regarding the profile of HBsAg, HBc and CD9, no differences were noticed as compared to the WT clone.

Next, 5′RACE reactions (Stadelmayer et al., 2020) were performed on: a) cell extracts from PLC/PRF/5 and Hep3B cells, known to express mostly S RNAs and, possibly, in the case of the Hep3B, HBx transcripts; b) cell extracts and the cell supernatants of Huh7-WT18 and Huh7-3D29 cell lines as well as of tet-off induced HepAD38 cells as a positive control. S transcripts were detected in PLC/PRF/5 and Hep3B cells and HBx transcripts in Hep3B cells, thus validating the 5′RACE performance and specificity. 3.5 kb RNA species were detected only in the supernatant of Huh7-3D29 cells, thus confirming the Nanopore sequencing results.

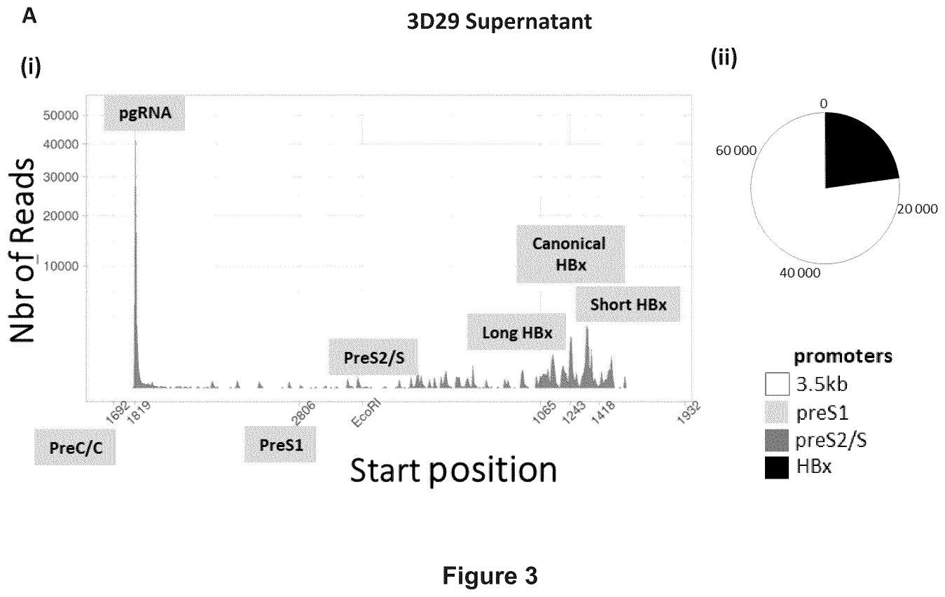

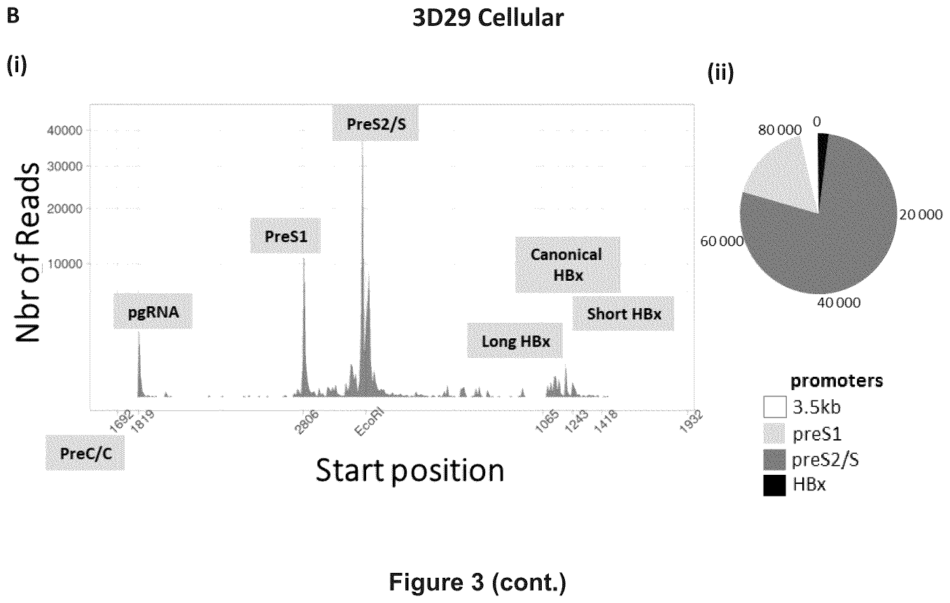

Finally, to further characterize the RNA species secreted by the Huh7-WT18 and mutant Huh7-3D29 clone single molecule long reads Nanopore sequencing of 5′RACE products was performed (FIG. 3). The sequencing results show that: a) the secreted HBV RNA is completely identical to the HBV genome used for the establishment of the different cell lines; b) the majority of HBV RNAs detected in cell supernatants are 3.5 KB full length pgRNA transcripts and pgRNA-derived spliced RNAs whereas only a minority of PreS/S RNAs, PreS/S derived spliced RNA and HBx RNAs were detected (panel A). The comparison with the HBV RNA species detected in the intracellular compartments highlights the selective secretion of 3.5 kB HBV RNAs vs preS/S transcripts (Panel B); c) all reads terminate at the canonical poly-adenylation site RNAs.

As a conclusion, the Huh7-3D29 maintained its RNA secretory phenotype with an extremely high RNA/DNA ratio in cell supernatants with negligible residual DNA. If one considers the performing characteristics of the Roche MWR used in this study (or the related HBV RNA cobas® 6800/8800 automated investigational assay) that tolerates DNA:RNA ratios up to approximately 10⁶ without losing specificity and linearity, the HBV RNA standard produced and secreted by the Huh7-3D29 cell line fulfills this criterion. Notably, HBV RNA quantification of Huh7-3D29 supernatants with the Roche MWF showed no significant difference with and without DNase treatment, supporting the use of the Huh7-3D29 derived HBV RNA standard with both automated and manual assays. Additional observations validate the use of Huh7-3D29 cell supernatants as an HBV RNA standard. First, the quantification of HBV RNAs with the Roche MWF of standard curves obtained from 10-fold dilution of Huh7-3D29 supernatants and a synthetic HBV armored RNA showed the same profile. Second, the CTs obtained with both standards showed no significant differences. Third, the RNA production and secretion in Huh7-3D29 cells show a good degree of concordance to what observed in vivo in chronic HBV carriers. The results of iodixanol/sucrose gradients indicate that the majority of secreted HBV RNAs are encapsidated and are found in naked capsid and in virions-like particle. Single molecule long reads Nanopore sequencing indicates that the majority of the Huh7-3D29 secreted HBV RNAs are full length or spliced 3.5 KB species and most, if not all, RNAs terminate at the canonical HBV polyA site. These results support the potential use of Huh7-3D29 supernatants as a standard for all HBV RNA investigational assays and in house PCR-based HBV RNA assays.

According to our results, the HBV RNA quantities produced from one 175 cm² flask of Huh7-3D29 clonal cell line in 9 days provides enough material for 1,300 standard curves using 20 μL of 10⁶ copies/mL preparations. Therefore, the Huh7-3D29 cell line constitutes a potentially unlimited source of HBV RNA standard. Importantly, the fact that the vast majority of HBV RNAs detected in 3D29 supernatants are 3.5 KB full length pgRNA transcripts, support the use of the 3D29 HBV RNA standard with all HBV RNA assays, including the existing investigational assays (Butler 2018; Scholtes 2022) and in-house research use only ddPCR assays as well as all future tests based upon PCR technologies.

### References

Baylis, S. A., Heath, A. B., Chudy, M., Pisani, G., Klotz, A., Kerby, S., and Gerlich, W. (2008). An international collaborative study to establish the 2nd World Health Organization International Standard for hepatitis B virus DNA nucleic acid amplification technology-based assays. Vox Sanguinis 94, 358-362.
Butler, E. K., Gersch, J., Mcnamara, A., Luk, K .-C., Holzmayer, V., de Medina, M., Schiff, E., Kuhns, M., and Cloherty, G. A. (2018). Hepatitis B Virus Serum DNA andrna Levels in Nucleos(t)ide Analog-Treated or Untreated Patients During Chronic and Acute Infection. Hepatology 68, 2106-2117.
Fryer, J. F., Heath, A. B., Wilkinson, D. E., Minor, P. D., and Collaborative Study Group (2017). A collaborative study to establish the 3rd WHO International Standard for hepatitis B virus for nucleic acid amplification techniques. Biologicals 46, 57-63.
Heermann, K .-H., Gerlich, W. H., Chudy, M., Schaefer, S., and Thomssen, R. (1999). Quantitative Detection of Hepatitis B Virus DNA in Two International Reference Plasma Preparations. J Clin Microbiol 37, 68-73.
Jenkins, A., Minhas, R., Morris, C., and Berry, N. (2017). Complete Genome Sequence of the WHO International Standard for Hepatitis B Virus DNA. Genome Announc. 5.
Klymiuk, M. C., Balz, N., Elashry, M. I., Heimann, M., Wenisch, S., and Arnhold, S. (2019). Exosomes isolation and identification from equine mesenchymal stem cells. BMC Veterinary Research 15, 42.
Liu, S., Deng, R., Zhou, B., and Sun, J. (2020). Is World Health Organization HBV DNA Standard Appropriate for Standardizing Serum HBV RNA Assay? Clin Infect Dis.
Organization, W.H., and Standardization, W.E.C. on B. (2016). Collaborative study to evaluate the proposed WHO 4th International Standard for Hepatitis B Virus (HBV) DNA for Nucleic Acid Amplification Technique (NAT) based assays.
Saldanha, J., Gerlich, W., Lelie, N., Dawson, P., Heermann, K., Heath, A., and WHO Collaborative Study Group (2001). An international collaborative study to establish a World Health Organization international standard for hepatitis B virus DNA nucleic acid amplification techniques. Vox Sang 80, 63-71.
Scholtès C, Hamilton T A, Plissonnier M L, Charre C, Scott B, Wang L, Berby L, French J, Testoni B, Blair. A, Subic M, Hoppler M, Lankenau A, Grubenmann A, Levrero M, Heil M L, Zoulim F. (2022) Performance of the cobas® HBV RNA Automated Investigational Assay for the Detection and Quantification of Circulating HBV RNA in Chronic HBV Patients. J Clin Virol 150-151:105150
Stadelmayer, B., Diederichs, A., Chapus, F., Rivoire, M., Neveu, G., Alam, A., Fraisse, L., Carter, K., Testoni, B., and Zoulim, F. (2020). Full-length 5′RACE identifies all major HBV transcripts in HBV-infected hepatocytes and patient serum. J. Hepatol. 73, 40-51.

1. A cell line derived from a liver cancer cell line engineered to produce and secrete viral particles containing pregenomic RNA of the hepatitis B virus (HBV), predominantly over HBV DNA.

2. The cell line of claim 1, derived from a Huh7 hepatocarcinoma cell line.

3. The cell line of claim 1, wherein the cell line has been generated by transfection of a liver cancer cell line with a vector, preferably a plasmid vector, that comprises at least 1.1×HBV DNA genome carrying mutations that alter viral DNA synthesis but preserve pregenomic RNA synthesis and viral protein expression, resulting in the production and secretion of viral particles containing pregenomic RNA of HBV without substantial secretion of viral DNA containing particles.

4. The cell line of claim 3, wherein the mutations comprise, or consist, of i) D540A and D541A amino acid change in the catalytic domain of Pol ORF, and ii) Y63F amino acid change in the TP-domain of the Pol ORF.

5. The cell line of claim 1, wherein the ratio of HBV secreted RNA/HBV DNA is at least 25/1.

6. The cell line of claim 1, deposited at CNCM on Aug. 1, 2022 under deposit number CNCM I-5878.

7. A vector that comprises at least a 1.1×HBV DNA genome carrying mutations that alter viral DNA synthesis but preserve pregenomic RNA synthesis and viral protein expression, preferably wherein the mutations comprise, or consist, of i) D540A and D541A mutations in the catalytic region of Pol ORF, and ii) Y63F mutation in the TP-domain of the Pol ORF.

8. The vector of claim 7, that is a plasmid vector.

9. A method for producing HBV RNA particles, which method comprises i) culturing the cell line as defined in claim 1 under conditions that allow the cells to secrete the HBV RNA particles, and ii) collecting the supernatant(s), that comprise HBV RNA particles.

10. The supernatant or collection of supernatants obtained or obtainable by the method of claim 9.

11. A method for calibration of circulating HBV RNA quantification assays, which method uses the supernatant or collection of supernatants as defined in claim 10 as a standard for calibration.

12. An in vitro method for diagnosing or monitoring an infection by HBV, for diagnosing or monitoring an infection by HBV, assessing or monitoring a HBV treatment, or for predicting patient outcome, which method comprises determining or quantifying HBV RNA by means of the supernatant or collection of supernatants of claim 10.

13. A kit for calibrating HBV RNA assays, comprising at least one container with HBV RNA containing particles in a supernatant or collection of supernatants as defined in claim 10.

14. The kit of claim 13, wherein the HBV RNA containing particles are in a concentration of at least 10⁷ copies/ml.

15. An in vitro method for diagnosing or monitoring an infection by HBV, for diagnosing or monitoring an infection by HBV, assessing or monitoring a HBV treatment, or for predicting patient outcome, which method comprises determining or quantifying HBV RNA by means of the supernatant or collection of supernatants of the kit of claim 13.
